(19) United States
(12) Patent Application Publication
(10) Pub. No.: US 2013/0337570 A1
(43) Pub. Date: Dec. 19, 2013

(54) METHOD FOR QUANTITATIVE ANALYSIS OF SUGARS, SUGAR ALCOHOLS AND RELATED DEHYDRATION PRODUCTS

(71) Applicants: Stephen Howard, Sherman, IL (US); Erik Hagberg, Decatur, IL (US); Erin Rockafellow, Decatur, IL (US)

(72) Inventors: Stephen Howard, Sherman, IL (US); Erik Hagberg, Decatur, IL (US); Erin Rockafellow, Decatur, IL (US)

(73) Assignee: Archer Daniels Midland Company, Decatur, IL (US)

(21) Appl. No.: 14/002,157

(22) PCT Filed: Feb. 27, 2012
(86) PCT No.: PCT/US12/26707

§ 371 Date: Aug. 29, 2013

Related U.S. Application Data
(60) Provisional application No. 61/451,380, filed on Mar. 10, 2011.

(51) Int. Cl. G01N 33/98; G01N 33/50

(52) U.S. Cl. CPC G01N 33/98; G01N 33/50
USPC 436/94

(57) ABSTRACT

An improved method is provided for the quantitative analysis of mixtures including various sugars, sugar alcohols and related dehydration products, whereby these are enabled to be effectively and accurately quantitated through gas chromatography, for example, by their derivatization with a carboxylic acid, carboxylic acid anhydride or halide in the presence of a metal triflate catalyst. The method can be carried out at essentially room temperature conditions, with a sufficiently rapid and complete derivatization, even in the presence of substantial amounts of water, that the materials to be quantitated do not substantially break down or degrade and substantially completely accounted for in a derivatized form.

Mixtures of sugars, sugar alcohols and/or of related dehydration products are encountered in various contexts, for example, in biochemical and food science contexts, as well as in other settings such as in the production of various chemical, fuel and fuel additive products from renewable, carbohydrate-based raw materials.

In a food science context, for instance, a knowledge of the qualitative and quantitative distribution of sugars in fruits, vegetables, honeys and other natural matrices is important for understanding qualities such as maturity, ripeness, authenticity and flavor and the effect of different harvesting, transportation, storage and processing details on such qualities.

In the context of producing various renewable source-based chemical, fuel and fuel additive products according to processes which oftentimes are based at least in part on naturally-occurring carbohydrates, the capacity to both qualitatively and quantitatively characterize feedstocks, products and byproducts is as important as in the petrochemical and petrofuels industries, for example, for process design, catalyst development, separation systems design, equipment sizing and for other purposes.

In a biochemical context, for instance, U.S. Pat. No. 6,309,852 describes the importance of being able to accurately quantitatively determine a specific component, 1,5-anhydroglucitol, in biological samples containing glucose, in diagnosing diabetes.

Unfortunately, because these same sugars, sugar alcohols and dehydration products are susceptible to thermal degradation or are too easily volatilized, the analytical tools and methods available to qualitatively and quantitatively characterize a given mixture have been limited, and the data produced—often laboriously and with certain precautionary measures being necessary—have been sometimes open to question or more difficult to properly assess.

In Chen et al., “Studies on Quantitative Analysis of Whole-Cell Sugars in Actinomycetes by Gas Chromatography-Mass Spectrum”, Weishengwexue Tongbao, vol. 27, no. 6, pp. 416-421 (2007), for example, the limitations of conventional analytical methods for quantitative determination of whole-cell sugars in actinomycetes bacteria are discussed, and an alternative approach is proposed for esterifying the sugars to sugar acetates and then analyzing by a combination of gas chromatography/mass spectroscopy (GC/MS).

Derivatization of certain species of interest, in order to make the species more amenable to analysis by a given method or methods, is a well-established concept in analytical chemistry. In the reference just cited, for example, sugars and sugar alcohols were acetylated over a period of three hours in an oscillating shaker at 55 degrees Celsius, using acetic anhydride in the presence of methylimidazole catalyst. Other, previous derivatization efforts are mentioned, wherein sugar trimethylsilyl ether derivatives were made but found less than satisfactory.

While the derivatization method of the Chen et al. reference was thus presented as an improvement, permitting a “significant increase in the speed of quantitative chemical analysis of sugars” in actinomycetes and “greatly increased” sensitivity, for the sugars, sugar alcohols and related dehydration products with which the present invention is concerned there are still aspects of Chen et al's method that provide room for improvement.

In particular, it would be beneficial—for diagnostic and corrective/process control purposes, for example—if the derivatization and subsequent analysis could be completed in a much shorter period of time, ideally, in a manner of minutes, not hours. As well, sugars and especially sugar dehydration products are not particularly stable at even moderate temperatures under acidic conditions, and in the case of sugar dehydration products one would risk causing their decomposition with an imidazole catalyst as favored by Chen et al. It would consequently be beneficial to derivatize the materials to be quantitated at even room temperature conditions, and further, to use a catalyst and method that are well-suited to the quantitative analysis of sugars, sugar alcohols (including monools, diols and polyols) and related dehydration products (e.g, isosorbide from sorbitol) alike. Finally, it is noted that Chen et al's method is dependent on a significant excess of acetic anhydride being present relative to any water in the sample—and since the samples routinely encountered in actual practice do often contain significant amounts of water—it would be beneficial if the catalyst and method were not so constrained.

The present invention against this background provides an improved method for the quantitative analysis of mixtures including various sugars, sugar alcohols and related dehydration products, whereby these are enabled to be effectively and accurately quantitated by their derivatization with a carboxylic acid, carboxylic acid anhydride or halide in the presence of a metal triflate catalyst, and then analyzing for the derivatives. In certain embodiments, the method is carried out at essentially room temperature conditions, namely, about 20 to 25 degrees Celsius. In the same or other embodiments, the derivatization is substantially completed (meaning, no substantial quantities of underivatized sugars, sugar alcohols or related dehydration products, as the case may be) before any appreciable degradation of the sugars, sugar alcohols or dehydration products to be quantitated occurs. And, in the same or other embodiments yet again, the method is employed on samples containing significant amounts of water, for example, about 50 volume percent of water and greater.

The present invention is in preferred embodiments directed to the quantitative analysis of mixtures including various sugars, or including various sugar alcohols, or including various related dehydration products, or including materials from two or all three of these categories. Particularly, through a metal triflate-catalyzed derivatization of these compounds in the mixtures through reacting with a suitable carboxylic acid, carboxylic acid anhydride or halide, conventional gas or liquid chromatographic methods can be used (as appropriate, with other common analytical techniques such as mass spectroscopy or a UV spectrophotometry, for example) to effectively and accurately characterize and quantify the amounts of underivatized materials of interest in the original sample matrix.

The particular carboxylic acid, carboxylic acid anhydride or halide used, of course, will be selected to provide derivatives which readily lend themselves to an accurate characterization and quantification by the analytical method or methods of choice; acetic anhydride as used by Chen et al., for instance, provides acetyl derivatives which are readily analyzed by gas chromatography, though it is understood that other reactants (for example, other acid anhydrides) could also be used with gas chromatography or other conventional analytical methods. Derivatization with benzoic anhydride would yield derivatives that would be readily analyzed by a combination of liquid chromatography and ultraviolet spectrometry, as an example of another acid anhydride and of other common analytical techniques that may be contemplated. The determination of an appropriate combination of analytical methods and derivatization reactants in the context of the present invention will, in any event, be well within the capabilities of those skilled in the art given the present description including the examples which follow hereafter

One exemplary application of the method of the present invention, demonstrated in Example 1, is for the quantitative analysis of isosorbide in an aqueous mixture. Isosorbide, a high boiling diol derived from the dehydration of sorbitol, has a variety of established and developing end uses and is conventionally sold in a flaked form or as an 85% mixture with water. A compositional analysis of isosorbide, as might be performed for example for quality assurance purposes, typically has required the application of liquid chromatography because many of the impurities which can be found in commercial isosorbide grades decompose or do not elute during gas chromatography. By acetylation with acetic anhydride in the presence of a bismuth triflate catalyst as shown in Example 1 below, however, gas chromatographic methods were able to be successfully used with an 85% isosorbide/15% water mixture.

Another exemplary application of the method of the present invention is demonstrated in Example 2, to fully characterize and quantify the various sugars from the enzymatic or acid hydrolysis of a common biomass, corn stover. Those familiar with the efforts which have been underway for some time to synthesize renewable source-based chemical products, fuel and fuel additive products from biomass feedstocks (such as corn stover and other agricultural residues) will appreciate that many of these efforts have incorporated such a hydrolysis step as a means to fractionate a lignocellulosic biomass for further processing.

By way of background, lignocellulosic biomasses are comprised mainly of cellulose, hemicellulose and lignin fractions, with cellulose being the largest of these three components. Cellulose derives from the structural tissue of plants, and consists of long chains of beta glucosidic residues linked through the 1,4 positions. These linkages cause the cellulose to have a high crystallinity and thus a low accessibility to the enzymes or acid catalysts which have been suggested for hydrolyzing the cellulose to C6 sugars or hexoses for further processing. Hemicellulose by contrast is an amorphous heteropolymer which is easily hydrolyzed, while lignin, an aromatic three-dimensional polymer, is interspersed among the cellulose and hemicellulose within a plant fiber cell and lends itself to still other process options.

Because of the differences in the cellulosic, hemicellulosic and lignin fractions of biomass, as well as considering other lesser fractions present in various biomasses to different degrees, as related in U.S. Pat. No. 5,562,777 to Farone et al., “Method of Producing Sugars Using Strong Acid Hydrolysis of Cellulosic and Hemicellulosic Materials”, a number of processes have been developed or proposed over the years to fractionate lignocellulosic biomasses and hydrolyze the cellulosic and hemicellulosic fractions.

Fundamentally both biological and non-biological processes have been disclosed, with the oldest and best known non-biological methods of producing sugars from cellulose involving acid hydrolysis, most commonly sulfuric acid-based hydrolysis using a dilute acid approach, a concentrated acid approach or a combination of the two. The '777 patent to Farone et al.

describes the advantages and disadvantages of the various sulfuric acid-based processes then known to the art, and suggests a further variation using strong acid/sulfuric acid hydrolysis and employing one or more iterations of a combination of a decrystallization step wherein the biomass (and/or including the solids left from the decrystallization step in a previous iteration) is mixed with a 25-90 percent sulfuric acid solution to solubilize a portion of the biomass, then the acid is diluted to between 20 and 30 percent and the mixture heated to preferably between 80 and 100 degrees Celsius for a time to solubilize the cellulosic fraction and any hemicellulosic material that had not been hydrolyzed.

Additional exemplary biomass fractionation methods including some form (or forms) of acid hydrolysis, subsequent to the '777 Farone patent, are related in commonly-assigned Patent Cooperation Treaty Application Serial

No. PCT/US2011/02200, filed Jan. 21, 2011 for “Improved Process for Fractionating Biomass”, which application is now incorporated herein by reference, wherein concentrated organic acid vapors containing at least 50 percent by weight of one or more of acetic, propionic, malic, succinic, formic and lactic acids are applied to the biomass at elevated temperatures to at least partly depolymerize/solubilize the hemicellulosic and lignin materials in the biomass, and in commonly-assigned Patent Cooperation Treaty Application Serial No. PCT/US2011/021518, filed Jan. 16, 2011 for “Method of Producing Sugars Using a Combination of Acids to Selectively Hydrolyze Hemicellulosic and Cellulosic Materials”, now also incorporated herein by reference, wherein a first, comparatively weaker organic acid (such as acetic acid or formic acid) is applied to a biomass for providing a pentose product or stream, and a second, strong mineral acid (such as sulfuric acid) is subsequently applied for providing a separate hexose product or stream from hydrolyzing cellulosic materials in the biomass.

Numerous other examples of the application of enzymatic or acid hydrolysis to a biomass could be cited, of course, but it is considered that the method of the present invention will be especially well-suited to those processes using an acid or acids to hydrolyze biomass fractions and produce mixed sugar streams for subsequent fermentation to ethanol, for example, or for making other materials useful as a fuel additive, replacement fuel or renewable source-based replacement for a known petroleum-derived chemical product, since the resulting pentose and hexose sugars tend to quickly dehydrate under acidic conditions at even fairly moderate temperatures—and since the dehydration products tend to be even less stable.

As related above, the catalysts and method of the present invention are well-adapted to the quantitative analysis of mixtures including various sugars, sugar alcohols and related dehydration products, being particularly applied in a preferred embodiment to the substantially complete derivatization of the sugars, sugar alcohols and dehydration products which may be present in a sample at room temperature conditions of from about 20 to 25 degrees Celsius. Particularly where the sample is of an acidic character, or where degradation/decomposition of the sugar, sugar alcohol and/or dehydration products of interest is otherwise foreseeable, the catalysts of the present invention are sufficiently active even at room temperature conditions to enable the substantially complete derivatization to more stable derivatives, before any appreciable degradation can occur—for example, in the course of not more than 120 minutes, preferably in 60 minutes or less, more preferably in 30 minutes or less and most preferably 15 minutes or less. Further, even given the activity of the metal triflate catalysts of the present invention, the same catalysts have been found to be sufficiently selective to the acetylated derivatives that intermediates and byproducts do not appear to form to a degree whereby the sugars, sugar alcohols and dehydration products may not be essentially completely accounted for in the acetylated derivatives.

In any event, preferably by means of the present invention it will be possible to account for at least 90, more preferably at least 95 and most preferably 99 percent of the sugars, sugar alcohols and dehydration products present in a sample originally in the form of the acetylated derivatives of these, through conventional analytical methods, namely consisting of gas or liquid chromatography and mass spectroscopy.

Because the samples of interest in the various biochemical, food science and industrial contexts mentioned above will frequently contain significant amounts of water, for example, 50 volume percent and greater, the metal triflate catalysts used for the inventive method include any of the water-tolerant, Lewis acid metal triflate catalysts, for example, bismuth and neodymium triflates, as well as lanthanide triflates. Very small amounts of catalyst will typically be required, for example, as little catalyst as 0.05 percent by mass or even less, based on the carboxylic acid, carboxylic acid anhydride or halide used for the acetylation. These triflate catalysts can be employed as is and recovered by washing the crude product with water, followed by evaporating the water, as demonstrated by the examples below. The catalyst may also precipitate out and be recovered at least in part by filtration, or the triflate catalyst might be incorporated on or into a solid substrate and recovered again by filtering rather than extraction; those skilled in the art will be well able to determine an appropriate method by which the Lewis acid metal triflate catalyst can be present in the system and subsequently recovered on completion of the derivatization reaction(s) for reuse.

The acetyl group can be supplied for the derivitization by a carboxylic acid, carboxylic acid anhydride or halide. Di-, tri- and polycarboxylic acids, anhydrides and chlorides may also be used, but for ease of synthesis and analysis and for convenience, acetic anhydride was selected for the examples and found to work well.

The present invention is more particularly illustrated by the examples which follow:

### EXAMPLE 1

Derivatization of isosorbide with acetic anhydride and bismuth triflate catalyst Commercially available isosorbide (Technical grade, 85%, product # 100100) was obtained from Archer Daniels Midland Co. (Decatur, Ill.) and derivatized as follows: a 0.1 g sample of isosorbide was weighed into a scintillation vial and 1.0 mL of acetic anhydride was added. Bismuth triflate catalyst (0.001 g) was added and the vial was carefully swirled for 10 minutes. Vials were then loosely capped and allowed to incubate 1 h with occasional gentle swirling. After incubation, a 1.00 mL aliquot of the sample was diluted with 9.00 mL of ethyl acetate.

In order to test the effectiveness of the derivatization procedure in the presence of water, a second 0.1 g sample of isosorbide was diluted with 15 wt % water to produce 85 wt % isosorbide. A sample of the 85% isosorbide was derivatized by substantially the same manner as the undiluted isosorbide.

Samples were analyzed by gas chromatography on an Agilent 7890 GC equipped with an Agilent DB-5 column, a FID detector and a 5975C mass spectrometer. Samples were injected in splitless mode into an injector port held at 250° C. using helium carrier gas flowing at 45 mL/min at 17.448 psi pressure. The DB-5 column (30 m×250 micrometer×0.5 micrometer) was held at 70° C. for one minute, ramped at 20° C./min to 180° C., held for two minutes at 180° C., ramped 20° C./min to 280° C., and then held at 280° C. for one minute. . Effluent was split and one stream passed through an FID maintained at 280° C. with a helium flow of 30 mL/min and an air flow of 350 mL/min, with a 15 mL/min makeup flow. A second effluent stream passed through the MS detector operated in relative EMV mode with EM voltage of 1200. The sample threshold was set to 150. The MS source was operated at 230° C., with the MS quad operated at 150° C. Samples of the solvent and acetic anhydride were also run as controls and any peaks present in the control samples were disregarded. Mass fragments and area percentages obtained from each detector are reported in Table 1.

TABLE 1

| | | | | Acetylated Isosorbide with 15% | | |
|---|---|---|---|---|---|---|
| | Acetylated Isosorbide | | | water added | | |
| | | Area | Area | | Area | Area |
| | Mass | % | % | Mass | % | % |
| Compound | fragments | (MS) | (MS) | fragments | (MS) | (FID) |
| Unknown 1 | 100, 73, 70, 61, | 0.04 | 0.05 | 100, 88, 73, | 0.05 | 0.06 |
| | 54, 43 | | | 70, 61, 57, 54, | | |
| | | | | 43 | | |
| Unknown 2 | | 0.00 | 0.00 | X | 0.01 | 0.04 |
| Unknown 3 | 145, 103, 43 | 0.01 | 0.03 | 145, 116, 103, | 0.04 | 0.06 |
| | | | | 73, 61, 43 | | |
| Unknown 4 | 152, 110 | 0.02 | 0.00 | X | 0.00 | 0.00 |
| Unknown 5 | 128, 85, 69, 57, | 0.05 | 0.11 | 128, 98, 85, | 0.09 | 0.21 |
| | 43 | | | 69, 61, 57, 43 | | |
| Unknown 6 | 110, 103, 94, 86, | 0.05 | 0.10 | X | 0.02 | 0.04 |
| | 69, 60, 43 | | | | | |
| Unknown 7 | 94, 81, 70, 43 | 0.02 | 0.04 | X | 0.01 | 0.02 |
| Unknown 8 | 85, 69, 61, 43 | 0.02 | 0.06 | 128, 85, 69, | 0.01 | 0.08 |
| | | | | 60, 43 | | |
| Unknown 9 | | 0.00 | 0.00 | 143, 96, 83, | 0.03 | 0.07 |
| | | | | 61, 55, 43 | | |
| Unknown 10 | | 0.00 | 0.00 | 143, 96, 83, | 0.03 | 0.08 |
| | | | | 61, 55, 43 | | |
| Unknown 11 | 155, 99, 57, 41 | 0.01 | 0.06 | X | 0.00 | 0.04 |
| Unknown 12 | 127, 111, 86, 69, | 0.04 | 0.10 | 86, 69, 60, 43 | 0.02 | 0.05 |
| | 60, 43 | | | | | |
| Unknown 13 | X | 0.05 | 0.10 | 128, 110, 102, | 0 04 | 0.07 |
| | | | | 97, 85, 69, 43 | | |
| Isomannide | 170, 141, 127, | 0.29 | 0.43 | 170, 127, 115, | 0.09 | 0.00 |
| diacetate | 115, 110, 99, 85, | | | 110, 99, 85, | | |
| | 69, 61, 55, 43 | | | 69, 55, 43 | | |
| Isosorbide | 231, 187, 170, | 97.01 | 96.52 | 231, 187, 170, | 98.02 | 97.66 |
| diacetate | 141, 127, 117, | | | 141, 127, 117, | | |
| | 110, 99, 85, 69, | | | 110, 99, 85, | | |
| | 55, 43 | | | 69, 55, 43 | | |
| Isosorbide | 243, 170, 159, | 0 73 | 0.32 | 243, 170, 159, | 0.82 | 0.48 |
| monoacetate/ | 141, 127, 110, | | | 143, 127, 110, | | |
| monopropionate | 99, 85, 71, 55, | | | 99, 85, 71, 55, | | |
| | 43 | | | 43 | | |
| Isosidide | 170, 127, 115, | 0.14 | 0.06 | 170, 127, 115, | 0.29 | 0.00 |
| diactetate | 110, 99, 85, 69, | | | 110, 99, 85, | | |
| | 61, 55, 43 | | | 69, 60, 55, 43 | | |
| Unknown 14 | X | 0.22 | 0.07 | X | 0.00 | 0.00 |
| Unknown 15 | 129, 112, 70, 57, | 0.07 | 0.08 | 129, 110, 69, | 0.03 | 0.00 |
| | 43 | | | 60, 43 | | |
| Unknown 16 | | 0.03 | 0.03 | X | 0.04 | 0.04 |
| Unknown 17 | X | 0.03 | 0.02 | 127, 110, 85, | 0.03 | 0.04 |
| | | | | 69, 57, 43 | | |
| Unknown 18 | X | 0.04 | 0.08 | 155, 110, 95, | 0.01 | 0.03 |
| | | | | 69, 60, 43 | | |
| Unknown 19 | X | 0.04 | 0.08 | X | 0.01 | 0.05 |
| Unknown 20 | | 0.00 | 0.00 | | 0.00 | 0.01 |
| Unknown 21 | X | 0.01 | 0.05 | | 0.00 | 0.00 |
| Unknown 22 | X | 0.01 | 0.03 | | 0.00 | 0.02 |
| Sorbitan | 259, 212, 187, | 0.84 | 1.15 | 207, 187, 170, | 0.24 | 0.50 |
| tetraacetate | 170, 153, 145, | | | 153, 145, 139, | | |
| isomer 1 | 127, 115, 97, 85, | | | 127, 115, 110, | | |
| | 69, 43 | | | 103, 97, 85, | | |
| | | | | 69, 60, 43 | | |
| Sorbitan | X | 0.02 | 0.04 | | 0.00 | 0.03 |
| tetraacetate | | | | | | |
| isomer 2 | | | | | | |
| Sorbitan | X | 0.03 | 0.06 | | 0.00 | 0.04 |
| tetraacetate | | | | | | |
| isomer 3 | | | | | | |
| Sorbitan | 259, 187, 170, | 0.17 | 0.28 | 207, 170, 152, | 0.06 | 0.18 |
| tetraacetate | 152, 139, 128, | | | 139, 110, 97, | | |
| isomer 4 | 110, 97, 85, 69, | | | 85, 69, 60, 43 | | |
| | 60, 43 | | | | | |
| Unknown 23 | X | 0.01 | 0.04 | 207, 149, 44 | 0.01 | 0.08 |

X = peak present, but no mass spectrum obtained

Derivatization (acetylation) with bismuth triflate allowed sensitive quantification of isosorbide and a number of impurities by gas chromatography. Acetylation in the presence of bismuth triflate followed by analysis by GC detected and quantified 30 compounds in addition to being able to detect including isosorbide propionate, likely due to the presence of a propionate impurity in the acetic anhydride used.

In addition, the derivatization method was sufficiently robust that isosorbide was substantially quantitatively derivatized in the presence of water. Non-acetylated isosorbide would elute between Unknown 3 and Unknown 4, but no non-acetylated isosorbide peak was present. Conversely, under optimized conditions, GC analysis of the same isosorbide samples without acetylation was only able to identify 11 compounds.

### EXAMPLE 2

Acetylation of sugars in corn stover hydrolysate Chopped corn stover (1.1 kg) was heated with 5 L of 70% acetic acid (v/v in water) in a steam-jacketed tumbling reactor. The reactor was brought to 150° C. within 10-20 minutes. Heating was continued for an additional 20 minutes with a maximum temperature between 165-170° C. The liquid fraction containing hydrolyzed hemicellulose and dissolved lignin was separated from the cellulose pulp solids by vacuum filtration. The pulp was washed once with hot 70% acetic acid (4-6 L), filtered, and then washed once with hot water (4 L) and filtered. The liquid fraction and both filtrates were combined, and contained hydrolyzed hemicellulose, dissolved lignin, and pulp wash liquid; the combined liquid and filtrate solution was concentrated to 35-50% dry solids to form a concentrated syrup.

Lignin was then precipitated from the concentrated syrup as follows: three parts of water were added to one part concentrated syrup, the mixture was stirred for one hour in a washing step, and the mixture was allowed to settle overnight. Some lignin precipitated, and was removed by vacuum filtration. The filtrate was concentrated to 35-50% solids by evaporation to form a washed concentrated syrup. The washed concentrated syrup was counter-current extracted four times with methyltetrahydrofuran in a solvent extraction step by passing the aqueous syrup through the organic solvent in a separatory funnel to form an aqueous fraction comprising solvent-washed concentrated syrup. The aqueous fraction was collected and boiled to remove residual solvent. Charcoal powder was added to the hot boiled solvent-washed concentrated syrup and stirred, and then filtered. The final filtered aqueous fraction contained 25% solids and comprised a hydrolyzed aqueous hemicellulose fraction from corn stover hydrolysate.

Hydrolyzed aqueous hemicellulose fraction (2 g) was acid treated to fully hydrolyze sugar oligomers by mixing with 6% (w/w) sulfuric acid (4 g), partitioned into 2 mL aliquots, and the aliquots were heated in an autoclave at 132° C. for 10 minutes to form depolymerized hydrolyzed aqueous hemicellulose fraction. A sugar recovery standard containing 1.4% xylose, 0.5% glucose, 0.2% galactose, 0.1% arabinose, and 0.1% mannose (w/w) (roughly the expected composition of the hydrolyzed aqueous hemicellulose fraction) was acid treated in the same manner.

Acetylation catalyst was prepared as follows: bismuth triflate (98%, Strem Chemicals, 20-40 mg) was added to acetic anhydride (Aldrich, 1 mL) under nitrogen to form a catalyst solution. As the bismuth triflate dissolved, the catalyst solution became warm and turned yellow. After the catalyst solution cooled to room temperature, 100 μL of the depolymerized hydrolyzed aqueous hemicellulose fraction was cautiously added. The reaction temperature rose rapidly to 54° C. The mixture was stirred for 6 h to form acetyl derivatives of sugars in the depolymerized hydrolyzed aqueous hemicellulose fraction, and a solid precipitated. Solvent was removed under reduced pressure. The derivatized residue was diluted with water and extracted with methylene chloride, which produced an emulsion that was broken up by centrifugation, resulting in three layers: an organic phase, an aqueous phase, and a precipitated solid phase. The same procedure was repeated with the sugar standard. The organic, aqueous, and solid layers present after centrifuging in both samples were analyzed by TLC and GC-MS.

Fully acetylated sugars in the standard solution and in the acetylated corn stover hydrolysate sample were identified by mass spectra and comparison of retention times to a known sugar standard derivatized conventionally using acetic anhydride, pyridine, and heat without bismuth triflate catalyst (not shown).

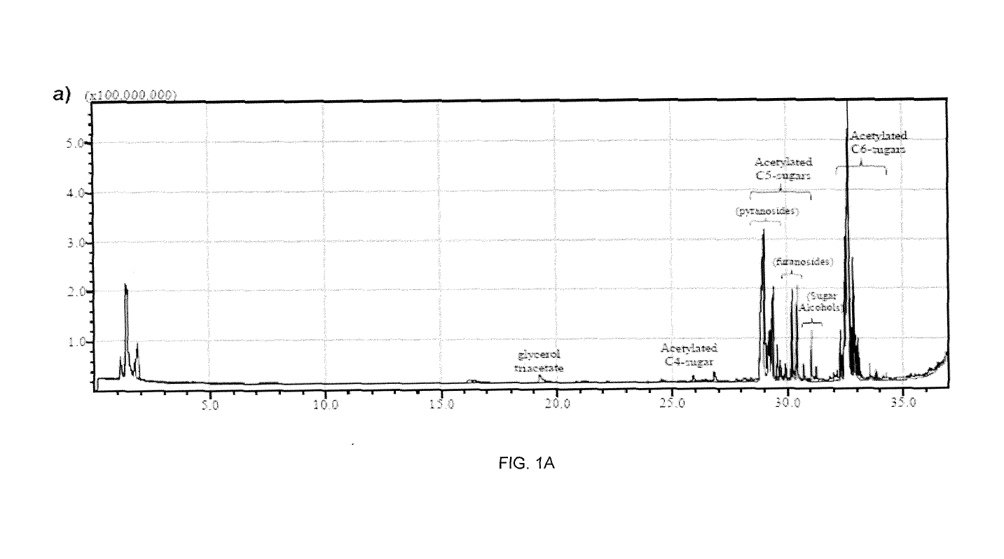

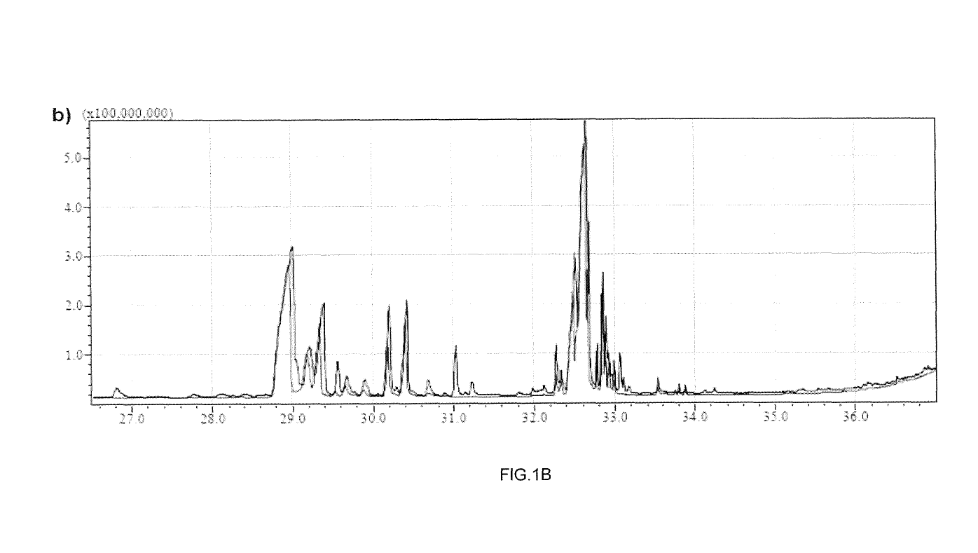

Derivatization catalyzed by bismuth triflate was sufficiently robust to derivatize the hydrolyzed aqueous hemicellulose fraction. Bismuth triflate catalyzed acetylation was very effective and proceeded to completion. Underivatized sugars were not detectable by TLC in any of the samples. In addition, GC-MS of the organic layer showed the presence of fully acetylated sugars and absence of partially acetylated sugars, including even trace amounts of acetylated sugar degradation products, in the acetylated corn stover hydrolysate sample (see FIGS. 1A and 1B accompanying, wherein FIG. 1A provides the full view of the GC-MS chromatogram of the organic layers from a bismuth triflate-catalyzed acetylation of corn stover hydrolyzate (shown in black) and a sugar standard (red), and FIG. 1B provides an enlarged view of the sugar region of the chromatogram).

Analysis of the aqueous layer showed only trace amounts of sugars (run as trimethylsilyl derivatives) with signals just above the chromatographic noise.

### EXAMPLE 3

Recovery and second use of bismuth triflate catalyst The solid that precipitated during the reaction in Example 2 was analyzed for metals by an inductively coupled plasma analytical method and contained only bismuth and sulfur. In order to determine if the precipitated catalyst was still active, the precipitated catalyst was tested in the acetylation of 2-propanol to produce isopropyl acetate. A few milligrams (spatula tip sized scoop) of the precipitated catalyst was added to acetic anhydride (1 mL) under nitrogen, producing a cloudy solution. After 10 min of stirring, isopropyl alcohol (100 μL) was added dropwise. The reaction temperature rose a few degrees, and began to fall. The mixture was stirred for 2.5 h at room temperature, and then centrifuged. A portion of the liquid was diluted with CD₂Cl₂ and analyzed by NMR. ¹H NMR of the reaction mixture revealed that the 2-propanol had been completely converted to isopropyl acetate, demonstrating that the catalyst was still active.

What is claimed is:

1. A method for the quantitative analysis of a mixture including a plurality of compounds selected from the sugars, sugar alcohols and the dehydration products of these, wherein the compounds are derivatized by reaction with a carboxylic acid, carboxylic acid anhydride or halide in the presence of a metal triflate catalyst, and the derivatized compounds then quantitatively analyzed.

2. A method according to claim 1, wherein the mixture contains 50 volume percent or more of water.

3. A method according to claim 1, wherein the derivatization proceeds substantially to completion in not more than 120 minutes.

4. A method according to claim 3, wherein the reaction proceeds substantially to completion in 60 minutes or less.

5. A method according to claim 4, wherein the reaction proceeds substantially to completion in 30 minutes or less.

6. A method according to claim 5, wherein the reaction proceeds substantially to completion in 15 minutes or less.

7. A method according to claim 3, wherein the derivatization is accomplished at from 20 to 25 degrees Celsius.

8. A method according to claim 3, wherein at least 90 percent of the sugars, sugar alcohols and dehydration products present in the underivatized mixture are accounted for in their acetylated forms by means of the quantitative analysis.

9. A method according to claim 8, wherein at least 95 percent of the sugars, sugar alcohols and dehydration products present in the underivatized mixture are accounted for in their acetylated forms by means of the quantitative analysis.

10. A method according to claim 8, wherein at least 99 percent of the sugars, sugar alcohols and dehydration products present in the underivatized mixture are accounted for in their acetylated forms by means of the quantitative analysis.

11. A method according to claim 1, wherein the quantitative analysis for the derivatized compounds is accomplished at least in part by gas chromatography.

12. A method according to claim 11, wherein the compounds are derivatized by reaction with acetic anhydride in the presence of a Lewis acid metal triflate catalyst.

13. A method for the quantitative analysis of isosorbide in an aqueous mixture, comprising acetylating the isosorbide through reaction with acetic anhydride in the presence of a Lewis acid metal triflate catalyst, and then quantitatively analyzing the mixture for the acetylated derivatives from isosorbide.

14. A method according to claim 13, wherein gas chromatography is used for the quantitative analysis.

15. A method according to claim 13, wherein a bismuth triflate catalyst is used.

16. A method according to claim 13, wherein the acetylation is carried out at room temperature to substantial completion.

17. A method according to claim 16, wherein substantial completion is accomplished in not more than 120 minutes.

18. A method for the quantitative analysis of sugars in a biomass hydrolyzate fraction from the enzymatic or acid hydrolysis of a lignocellulosic biomass, and the fractionation of the hydrolyzed biomass into cellulosic pulp solids, lignin and hydrolyzed hemicellulose fractions, comprising acetylating the sugars in the hydrolyzate fraction with acetic anhydride in the presence of a Lewis acid metal triflate catalyst, then quantitatively analyzing the mixture for the acetylated sugar derivatives.

19. A method according to claim 18, wherein the acetylation is carried out to substantial completion and under room temperature conditions with a bismuth triflate catalyst.
